(19) United States
(12) Patent Application Publication
(10) Pub. No.: US 2009/0216146 A1
(43) Pub. Date: Aug. 27, 2009

(54) Method and System for Processing and Electroencephalograph (Eeg) Signal

(76) Inventors: Mina Teicher, Tel Aviv (IL); Esther Adi-Japha, Rehovot (IL); Amir Zilberstein, Moshav Yad-Rambam (IL)

Correspondence Address: THE NATH LAW GROUP, 112 South West Street, Alexandria, VA, 22314, US

(21) Appl. No.: 11/988,366

(22) PCT Filed: Jul. 9, 2006
(86) PCT No.: PCT/IL06/00790

§ 371 Date: Mar. 30, 2009

Related U.S. Application Data
(60) Provisional application No. 60/696,809, filed on Jul. 7, 2005.

(51) Int. Cl. A61B 5/0476

(52) U.S. Cl. 600/544

(57) ABSTRACT

The invention provides a method and system for locating an epileptic focus in an individual. In accordance with the method of the invention, an integer N of ictal EEG signals are obtained on the individual and a principal component analysis generating N output signals is executed on the ictal EEG signals. The epileptic focus s then located in a process involving one or more of the N output signals.

### FIELD OF THE INVENTION

This invention relates to medical devices, and more specifically to such devices for recording and analyzing EEG signals.

### BACKGROUND OF THE INVENTION

Epilepsy is a brain disorder characterized by recurrent seizures resulting from abnormal electrical behavior of a population of brain cells known as the “epileptogenic region” or “epileptic focus”. This region is defined as the smallest area in the brain, whose removal results in a total cessation of the seizure (Engel et al., 1993). In some cases, the preferred treatment for epilepsy is surgical removal of the epileptic focus. This entails locating the epileptic focus, a process that usually relies on a combination of findings obtained by multiple techniques.

Using EEG recordings to locate an epileptic focus is advantageous due to its noninvasive nature and low cost. Locating an epileptic focus from an integer N of simultaneous ictal EEG signals involves calculating, for each of the N signals, a value of a parameter indicative of the similarity of the signal with a characteristic epileptic EEG signal. For example, each of the N signals may be subjected to band pass filtering at a frequency characteristic of an ictal EEG signal, and the amplitude of the filtered signal determined. As another example, the power of each signal in a given period (such as 2 secs.) of a selected typical seizure frequency may be calculated as the parameter (Blanke et al., 2000). Some computerized methods for locating the focus that utilize a full temporo-spatial data structure most frequently use short episodes of about 50 msec of the temporo-spatial EEG data in order to extract the relevant features of a typical spike. The various methods thus differ in the duration of the signals analyzed and the parameters extracted from the EEG signals. The N calculated values are input to an “inverse algorithm” which is defined as any algorithm that determines a location of an epileptic focus from the N input parameters. An inverse algorithm may be linear or non-linear. Linear inverse algorithms include such algorithms as “Minimum Norm Estimation” (MNE), and “Low Resolution Brain Electromagnetic Tomography” (LORETA). Linear inverse algorithms are reviewed in RD. Pascal-Marqui, 1999. A non-linear inverse algorithms based on genetic algorithms is presented in Zilberstein A., et al. 2002. Computational methods aimed at identifying relevant components of epilepsy in EEG recordings that are statistically independent from each other have been implemented, and topographic maps have been calculated for each component applied to time intervals of 80 seconds around seizure onset (Nam et al., 2002).

A difficulty in localizing an epileptic focus from scalp EEG recordings arises due to the presence of other generators in the brain whose activity masks, at least partially, the activity of the epileptic focus. or foci in the EEG recordings. The contribution of these generators to the EEG signals interferes with the analysis of the EEG signals that is a prerequisite for utilizing an inverse algorithm to locate an epileptic focus.

### SUMMARY OF THE INVENTION

In its first aspect, the present invention provides a method for processing an integer N of EEG signals. In accordance with the invention, a principal component analysis (PCA) is applied to the N signals. PCA is defined as any algorithm that transforms a set of vectors into an orthogonal coordinate system in which the first axis captures most of the variane of the vectors, the second axis captures most of the remaining variance, and so on. The PCA thus transforms the N EEG signals into N output signals. The N output signals are mutually orthogonal to each other and each obtained signal explains a different portion of the variance associating the original N signals. PCA methods are disclosed, for example, in Jolliffe, J. T., 2002.

The method of the invention may be used to process N ictal EEG signals prior to application of an inverse algorithm to the signals. Thus in its second aspect, the invention provides a method for locating an epileptic focus from an integer N of ictal signals. In accordance with this aspect of the invention, an integer N of ictal EEG signals are subjected to PCA. From among the N signals output from the PCA, a signal is selected most similar to an epileptic EEG signal. Methods for selecting a signal most similar to an epileptic EEG signal are known in the art. For each of the N original EEG signals, a value of a parameter is calculated indicative of the similarity of the EEG signal to the selected output signal. For example, the fraction of the variance of the particular input signal that is explained by the selected output signal can be calculated. The resulting N calculated parameters are input to an inverse algorithm so as to locate an epileptic focus, as explained above.

It will also be understood that the system according to the invention may be a suitably programmed computer. Likewise, the invention contemplates a computer program being readable by a computer for executing the method of the invention. The invention further contemplates a machine-readable memory tangibly embodying a program of instructions executable by the machine for executing the method of the invention.

Thus, in its first aspect, the invention provides a computer implemented method for processing an integer N of input EEG signals comprising executing on the N EEG signals a principal component analysis generating N output signals.

In its second aspect, the invention provides a computer implemented method for locating an epileptic focus comprising:
obtaining an integer N of ictal EEG signals;
executing on the integer N of ictal EEG signals a principal component analysis generating N output signals; and
locating the epileptic focus in a process involving one or more of the N output signals.

In its third aspect, the invention provides a system for processing an integer N of EEG signals comprising a processor configured to execute on the N EEG signals a principal component analysis generating N output signals.

In its fourth aspect, the invention provides a system for locating an epileptic focus comprising:
An integer N of electrodes obtaining an integer N of EEG signals; and
A processor configured to execute on the integer N of EEG signals a principal component analysis generating N output signals, and to locate the epileptic focus by a method involving the one or more of the n output signals.

In its fifth aspect, the invention provides a computer implemented program storage device readable by machine, tangibly embodying a program of instructions executable by the machine to perform method steps for processing an integer N of EEG signals comprising executing on the N EEG signals a principal component analysis generating N output signals.

In its sixth aspect, the invention provides a computer implemented computer program product comprising a computer useable medium having computer readable program code embodied therein for processing an integer N of EEG signals, the computer program product comprising executing on the N EEG signals a principal component analysis generating N output signals.

In its sixth aspect, the invention provides a computer implemented program storage device readable by machine, tangibly embodying a program of instructions executable by the machine to perform method steps for locating an epileptic focus comprising:
obtaining an integer N of ictal EEG signals; and
executing on the integer N of ictal EEG signals a principal component analysis generating N output signals.

In its seventh aspect, the invention provides a computer implemented computer program product comprising a computer useable medium having computer readable program code embodied therein for locating an epileptic focus, the computer program product comprising:
computer readable program code for causing the computer to execute on an integer N of EEG signals a principal component analysis generating N output signals.

In its eighth aspect, the invention provides a computer program comprising computer program code means for performing all the steps of the method of the invention when said program is run on a computer.

In its ninth aspect, the invention provides a computer program embodied on a computer readable medium, where the computer program comprises computer program code means for performing all the steps of the method of the invention when said program is run on a computer.

### BRIEF DESCRIPTION OF THE DRAWINGS

In order to understand the invention and to see how it may be carried out in practice, a preferred embodiment will now be described, by way of non-limiting example only, with reference to the accompanying drawings, in which:

FIG. 1 shows a system for obtaining and analyzing an integer N of EEG signals in accordance with one embodiment of the invention;

FIG. 2 shows a method for processing an integer N of EEG signals in accordance with one embodiment of the invention;

FIG. 3 shows a method for locating an epileptic focus in accordance with one embodiment of the invention;

FIG. 4a shows 20 EEG recordings of Subject 1, FIG. 4b shows 20 output signals from a PCA analysis of the 20 EEG signals, FIG. 4c shows a seizure component of Subject 1 selected from among the 20 output signals, and FIG. 4d shows a localization of the epileptic focus in Subject 1; and

FIG. 5a shows 20 EEG recordings of Subject 2, FIG. 5b shows 20 output signals from PCA analysis of the 20 EEG signals, FIG. 5c shows a seizure component selected from among the 20 output signals of Subject 2 and FIG. 5d shows a localization of the epileptic focus in Subject 2.

### DETAILED DESCRIPTION OF PREFERRED EMBODIMENTS

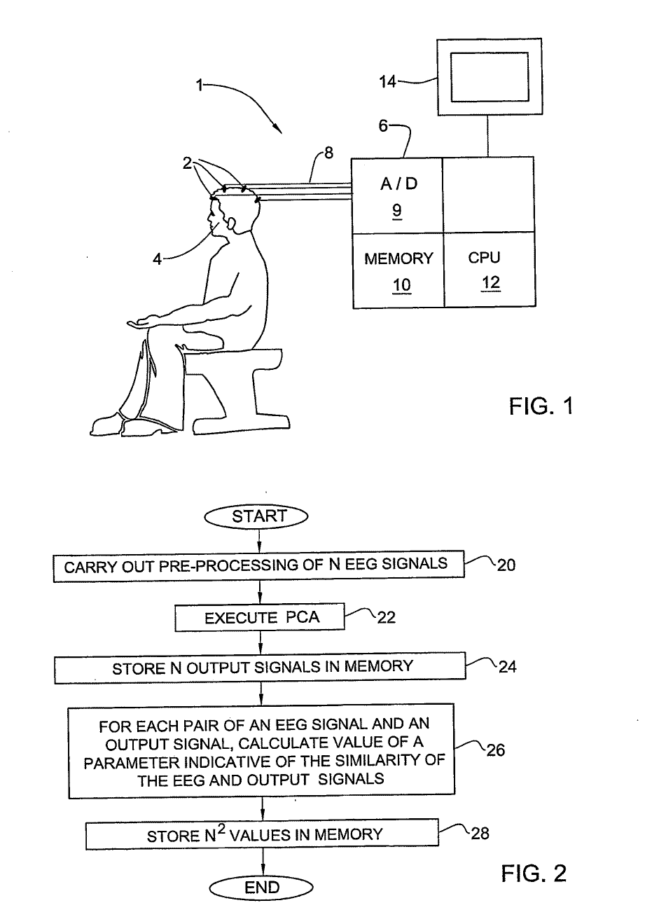

FIG. 1 shows a system 1 for obtaining and processing N EEG signals in accordance with one embodiment of the system of the invention. The system comprises N EEG electrodes 2 adapted to be attached to the scalp of a subject 4. Electrical signals 8 sensed by the electrodes are input to a processor 6 via cables. The processor includes an analog to digital converter 9. The processor 6 is configured to store digital data in a memory 10 associated with the processor 6.

The processor 6 includes a central processing unit (CPU) 12 configured to process the data. FIG. 2 shows a flow chart for a method of processing EEG signals in accordance with one embodiment of the method of the invention. In step 20, the CPU 12 pre-processes the signals 8. Pre-processing of the data may include, for example, filtering noise, band pass filtering the data in a frequency range characteristic of ictal EEG recordings, or detecting the onset of an epileptic seizure in one or more of the signals. In accordance with the invention, the processor is configured to execute a principal component analysis (PCA) on the N signals after any pre-processing. Thus, in step 22 the CPU 12 executes a PCA on the N signals. The result of the PCA is N output signals that are stored in the memory 8 (step 24). The PCA may also output, for each pair of an input signal and an output signal, a value of a parameter indicative of the similarity of the output signal to the input EEG signal (step 26). For example, the CPU 12 may calculate the fraction of the variance of the particular input signal that is explained by the output signal. The N² calculated values are stored in the memory 10.

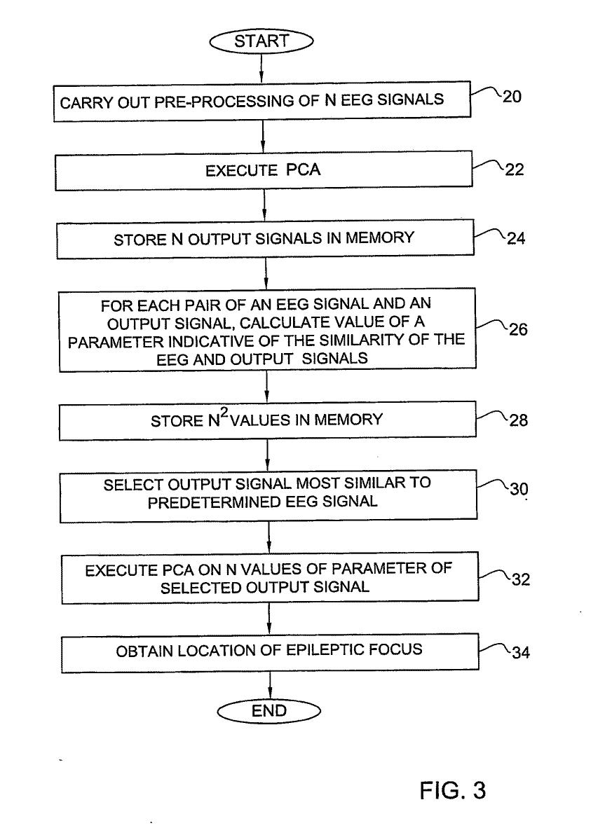

In a preferred embodiment of the invention, the CPU is further configured to locate an epileptic focus from the N input EEG signals 8. FIG. 3 shows a method for locating an epileptic focus. In step 20, the CPU 12 pre-processes the signals 8. Pre-processing of the data may include, for example, filtering noise, band pass filtering the data in a frequency range characteristic of ictal EEG recordings, or detecting the onset of an epileptic seizure in one or more of the signals. In accordance with the invention, the processor is configured to execute a principal component analysis (PCA) on the N signals after any pre-processing. Thus, in step 22 the CPU 12 executes a PCA on the N signals. The result of the PCA is N output signals that are stored in the memory 8 (step 24). The PCA may also output, for each pair of an input signal and an output signal, a value of a parameter indicative of the similarity of the output signal to the input EEG signal (step 26). For example, the CPU 12 may calculate the fraction of the variance of the particular input signal that is explained by the output signal. The N² calculated values are stored in the memory 10.

In step 30, one of the N output signals is identified that is most similar to a predetermined ictal EEG signal from among the N output signals. The output signal may be selected manually by displaying the output signals on a screen 14 (FIG. 1) associated with the processor 6 and determining visually which of the N output signals is most similar to the predetermined ictal EEG signal. Alternatively, the CPU 12 may be configured to select an output signal most similar to the predetermined ictal EEG signal.

In step 34 a PCA is executed by the CPU 12 on the N² values of the parameter that were calculated in step 26 for the selected output signal. The location of the epileptic focus is obtained from the PCA in step 34 and the process terminates.

### EXAMPLE

### Case Histories

Two case histories of temporal lobe focal epilepsy, as confirmed by a seizure free history following surgery of the focus, were selected for analysis. Table 1 shows the age, gender, seizure type, age of onset of seizures, and seizure frequency of the two subjects. Both subjects had been evaluated using scalp ictal and inter-ictal video-EEG, and brain magnetic resonance imaging (MRI). Seizures had been digitally recorded using 23 electrodes including 20 scalp EEG electrodes placed according to the 10/20 system as is known in the art, with a sampling rate of 200 Hz. Temporal lobectomy operations were performed at the Functional Neurosurgery unit at the Tel-Aviv Medical Center, Israel. Following the surgery, the patients were seizure free for at least two years.

### Identification of Ictal Epileptic Components

For each subject, the 20 EEG signals were filtered using a 0.1-70 Hz band pass filter. Onset of ictal activity in the 20 signals was identified independently by two readers, one of which was a “Board Certified EEGer”. The experts also determined the location of the epileptic focus from the data obtained by each of the three techniques that were used to evaluate the subjects. Table 1 shows the location of the epileptic focus as determined by the EEG experts. After identification of seizure onset by the experts, PCA analysis was applied to the 20 signals, over a time period of 5 sec starting from seizure onset. Using a duration of 5 sec minimizes the effect of noise resulting from the activity of generators in the brain unrelated to the epileptic focus. A PCA component representing seizure was extracted visually by selecting one of the 20 signals output by the PCA that explained most of the variance of the original N signals, and displayed a periodic characteristic of an ictal EEG signal (4-10 Hz) as identified by applying the FFT method (Blanke et al., 2000).

TABLE 1
Clinical information of patients
Location of epileptic focus

| | | | | Seizure | | | | |
|---|---|---|---|---|---|---|---|---|
| Sub- | Age | Gen- | Seizure | onset | Sz. | | Inter | |
| ject | (years) | der | type | (age) | Freq | Ictal | ictal | MRI |
| 1 | 38 | M | CPS, | 2 | 8/ | RT T | RT T | RT |
| | | | GTCS | | Month | | | MTS |
| 2 | 38 | F | CPS, | 28 | 6/ | RT T | RT T | RT |
| | | | GTCS | | Month | | | MTS |

Key:
CPS Complex Partial Seizures
GTCS Generalized Tonic-Clonic seizure
RT T Right Temporal
MTS Mesial Temporal Sclerosis

### Results

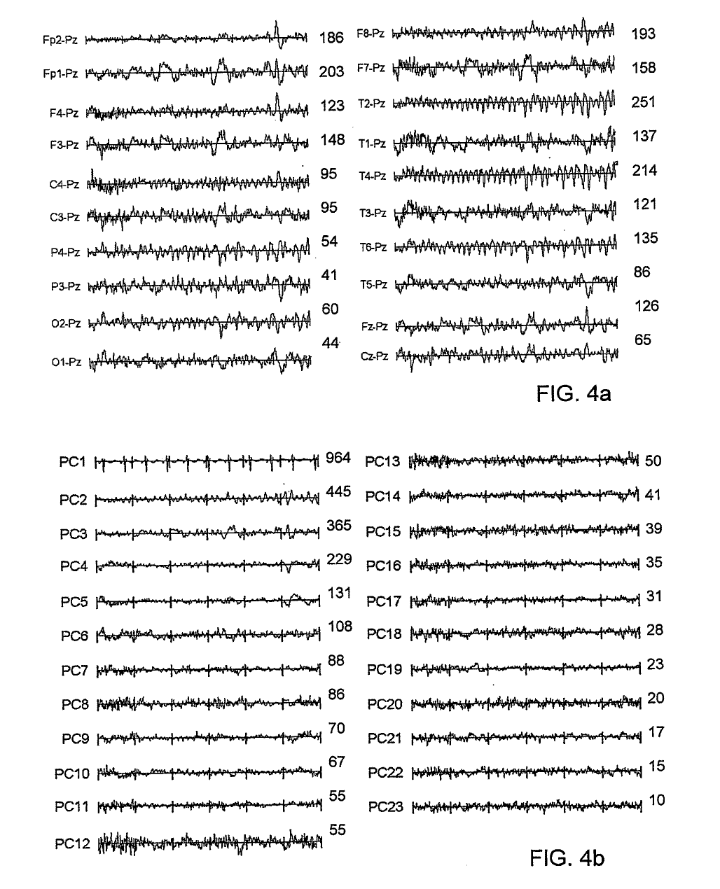

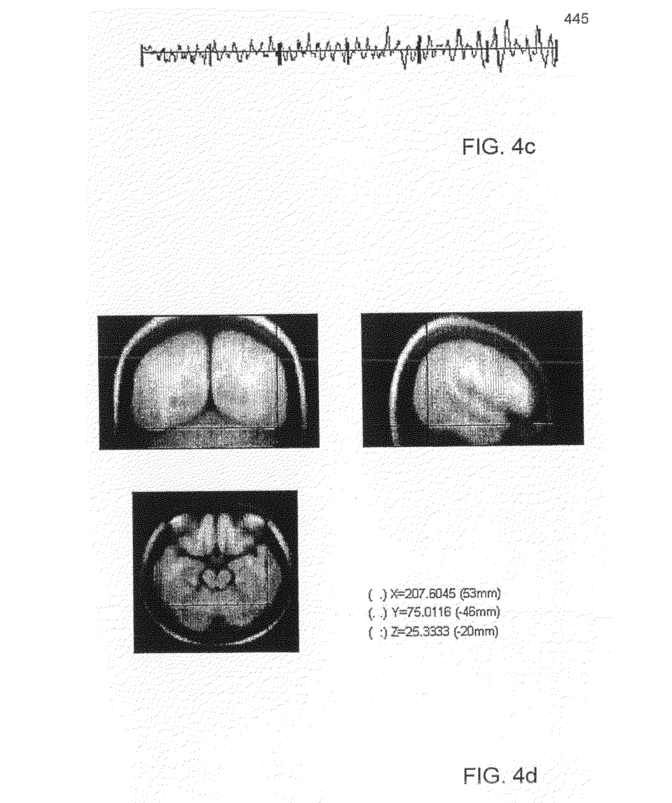

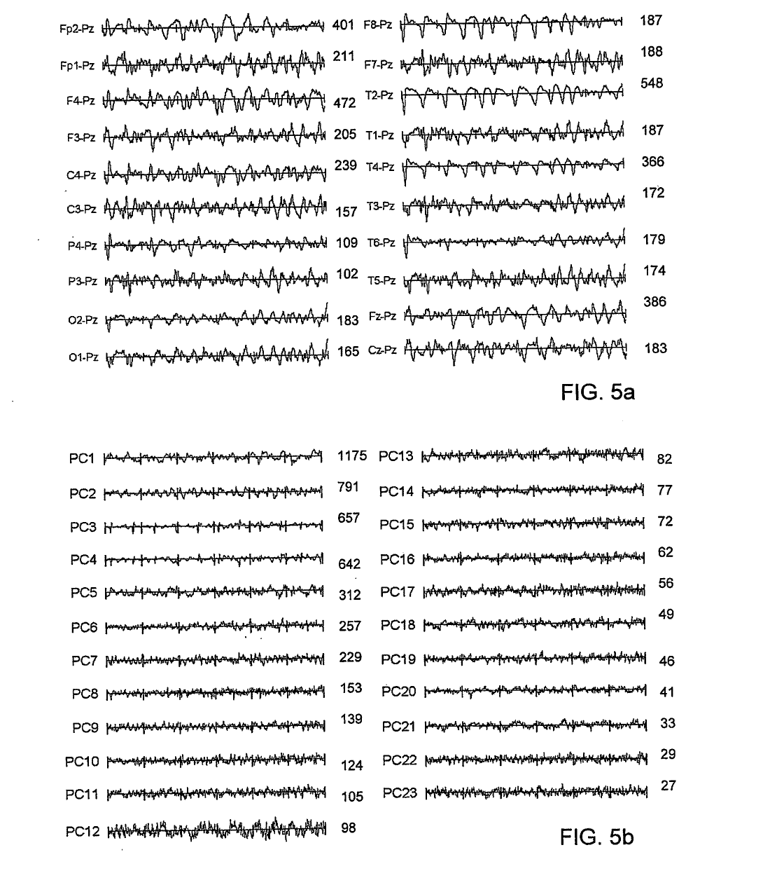

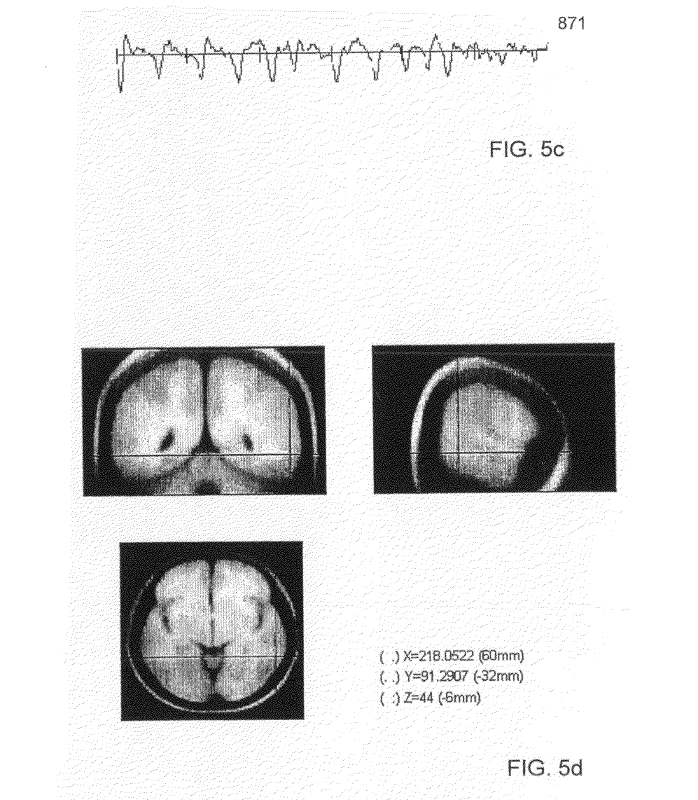

FIGS. 4a and 5a show the 20 recorded ictal EEG signals beginning at seizure onset as determined by the expert, for Subject 1 and 2 respectively. FIGS. 4b and 5b show the 20 signals output by the PCA analysis of the EEG signals of FIGS. 4a and 5a, respectively. FIGS. 4c and 5c show the seizure component for the Subject 1 and 2, respectively, that was selected from among the 20 components produced by the PCA. In both subjects, this component was the dominant first or second output signal of the PCA. The number in FIGS. 4a and 5a at the end of each of the EEG signals is the coefficient of the selected output signal component in the EEG signal. This coefficient is a parameter indicative of the similarity of the original EEG signal with the selected output signal.

### Localization of the Epileptic Focus

The vector of the 20 coefficients of the selected PCA component of each of the 20 original signals was input to a linear inverse algorithm. The algorithm calculates the location (x, y, z) of the source of the rhythmic activity displayed by the selected signal. Both the MNE and LORETA algorithms produced the same results. The coordinates of the calculated epileptic focus in both subjects is shown in Table 2 in the row of 0 sec from onset. The coordinates are in mm using the coordinate system of the Talairach Brain Atlas of the Brain Imagining Center at the Montreal Neurology Institute.

FIGS. 4d and 5d show the epileptic focus in the Subject 1 and 2, respectively, of the seizure as determined by the inverse algorithm. In both subjects the method of the invention accurately identified the epileptic focus as confirmed by the EEG experts and the subsequent surgery.

### Robustness Analysis

An analysis of the robustness of the method of the invention was performed by applying the method of the invention to 5 sec. intervals of the EEG starting within 5 sec. of seizure onset. In each analysis, a specific PCA output signal was selected as explained above and a corresponding location of the epileptic focus was calculated as described above. Table 2 presents the results of the analyses at the various starting times relative to seizure onset. In Table 2, “+” indicates that the localization of the focus determined by the method of the invention during the 5 sec interval indicated coincided with the localization calculated during the interval beginning with seizure onset. A “−” in Table 2 indicates that a different location was determined.

Table 2 shows that for both subjects, localization of the epileptic focus was found to be invariant under time shifts of 0.5 s in the start of the analyzed interval (relative to seizure onset). Time intervals starting far from seizure onset (5 sec. before or after seizure onset) do not allow accurate localization of the epileptic focus. For subject 2, all localization results calculated in an interval within ±2 sec of seizure onset were in agreement with the localization calculated from the interval beginning at seizure onset. For Subject 1, the localization results determined from intervals starting from −05 sec. to 2 sec. of seizure onset were in agreement with the localization calculated in the interval starting at seizure onset.

TABLE 2
Localization results of the inverse algorithm obtained for the component
representing epilepsy, calculated at different starting points relative to
seizure onset

| Time from | | | |
|---|---|---|---|
| onset | Duration | | |
| (sec) | (sec) | Subject 1 | Subject 2 |
| −5 | 5 | − | − |
| −2 | 5 | − | + |
| −1 | 5 | − | + |
| −0.5 | 5 | + | + |
| 0 | 5 | (53, −46, −20) | (60, −32, −6) |
| 0.5 | 5 | + | + |
| 1 | 5 | + | + |
| 2 | 5 | + | + |
| 5 | 5 | − | − |

### REFERENCES

Blanke et al. (2000). Temporal and spatial determination of EEG-seizure onset in the frequency domain. Clinical neurophysiology, 111, 763-772.
Engel J., Intracerebral recordings: organization of the human epileptogenic region. J. Clin Neurophysiol 1993; 10 (1): 90-98.
Jolliffe, J. T., 2002. Principal component Analysis, 2^{nd} Edition, Springer.
Nam et al., Epiplesia 43 (2): 160-164, 2002
Pascal-Marqui, International Journal of Bioelectromagnetism, 1 (1):75-86, 1999.
Zilberstein A. et al, Neuroplasticity 9 (2) p. 126, 2002.

1. A computer implemented method for processing an integer N of input EEG signals comprising executing on the N EEG signals a principal component analysis generating N output signals.

2. The method according to claim 1 further comprising, for each pair of an input EEG signal and an output signal, calculating a value of a parameter indicative of a similarity between the output signal and the EEG signal.

3. The method according to claim 2 wherein calculating a value of a parameter indicative of a similarity of an output signal with an EEG signal comprises calculating a variance of the EEG signal explained by the output signal.

4. A computer implemented method for locating an epileptic focus comprising:
(a) obtaining an integer N of ictal EEG signals;
(b) executing on the integer N of ictal EEG signals a principal component analysis generating N output signals; and
(c) locating the epileptic focus in a process involving one or more of the N output signals.

5. The method according to claim 4 wherein the step of locating the epileptic focus comprises:
(a) for each of one or more of the N EEG signals, determining a value of a parameter indicative of a similarity of a selected output signal with the EEG signal; and
(b) locating the epileptic focus by executing an inverse algorithm on the determined values of the parameter.

6. The method according to claim 5 wherein the selected output signal explains most of the variance of the N EEG signals.

7. The method according to claim 5, wherein the inverse algorithm is a linear inverse algorithm.

8. The method according to claim 5, wherein the inverse algorithm is a non-linear inverse algorithm.

9. The method according to claim 5, wherein the step of determining a value of a parameter indicative of a similarity of the selected output signal with an EEG signal comprises determining a variance of the EEG signal explained by the output signal.

10. A system for processing an integer N of EEG signals comprising a processor configured to execute on the N EEG signals a principal component analysis generating N output signals.

11. The system according to claim 10 wherein the processor is further configured, for each pair of an EEG signal and an output signal, to calculate a value of a parameter indicative of a similarity between the output signal and the EEG signal.

12. The system according to claim 11 wherein the processor is configured to calculate a value of a parameter indicative of a similarity of an output signal with a predetermined EEG signal by calculating a variance of the EEG signal explained by the output signal.

13. A system for locating an epileptic focus comprising:
(a) An integer N of electrodes obtaining an integer N of EEG signals; and
(b) A processor configured to execute on the integer N of EEG signals a principal component analysis generating N output signals, and to locate the epileptic focus by a method involving the one or more of the n output signals.

14. The system according to claim 13 wherein the processor is configured to locate the epileptic focus by a method comprising:
(a) for each of one or more of the N EEG signals, to determine a value of a parameter indicative of a similarity of a selected output signal with the EEG signal; and
(b) to locate the epileptic focus by executing an inverse algorithm on the determined values of the parameter.

15. The system according to claim 14, wherein the inverse algorithm is a linear inverse algorithm.

16. The system according to claim 14, wherein the inverse algorithm is a non-linear inverse algorithm.

17. The system according to claim 14, wherein the processor is configured to calculate a value of a parameter indicative of a similarity of an output signal with an EEG signal by calculating a variance of the EEG signal explained by the output signal.

18. A computer implemented program storage device readable by machine, tangibly embodying a program of instructions executable by the machine to perform method steps for processing an integer N of EEG signals comprising executing on the N EEG signals a principal component analysis generating N output signals.

19. A computer implemented computer program product comprising a computer useable medium having computer readable program code embodied therein for processing an integer N of EEG signals, the computer program product comprising executing on the N EEG signals a principal component analysis generating N output signals.

20. A computer implemented program storage device readable by machine, tangibly embodying a program of instructions executable by the machine to perform method steps for locating an epileptic focus comprising:
(a) obtaining an integer N of ictal EEG signals; and
(b) executing on the integer N of ictal EEG signals a principal component analysis generating N output signals.

21. A computer implemented computer program product comprising a computer useable medium having computer readable program code embodied therein for locating an epileptic focus, the computer program product comprising:
computer readable program code for causing the computer to execute on an integer N of EEG signals a principal component analysis generating N output signals.

22. A computer program comprising computer program code means for performing all the steps of claim 1 when said program is run on a computer.

23. A computer program as claimed in claim 22 embodied on a computer readable medium.
